(19) United States
(12) Patent Application Publication
(10) Pub. No.: US 2006/0247765 A1
(43) Pub. Date: Nov. 2, 2006

(54) Method of selecting an intraocular lens

(76) Inventor: Peter Fedor, Traverse City, MI (US)

Correspondence Address: Peter Fedor, MD, 9710 Peninsula Drive, Traverse City, MI, 49686, US

(21) Appl. No.: 11/120,458

(22) Filed: May 2, 2005

(51) Int. Cl. A61F 9/013

(52) U.S. Cl. 623/6.11; 128/898

(57) ABSTRACT

A method of selecting an intraocular lens, where the size, shape or overlap of capsulorhexis is used in addition to other factors to select the properties of the intraocular lens implanted in the eye to achieve certain refractive outcome.

### CROSS-REFERENCE TO RELATED APPLICATIONS

This application claims priority under 35 U.S.C. sctn. 19 from provisional patent application filed on May 3, 2004 by Peter Fedor, MD titled “Method of selecting an intraocular lens.”

### OTHER REFERENCES

U.S. patent Documents:

U.S. Pat. No. 5,282,852 February, 1994 Capetan et al Method of calculating the required power of an intraocular lens 632/6.

U.S. Pat. No. 5,968,095 October, 1999 Norby Method of selecting an intraocular lens to be implanted into an eye 632/6

### STATEMENT REGARDING FEDERALLY SPONSORED RESEARCH

Not Applicable

### BACKGROUND OF THE INVENTION

To obtain a desired refractive outcome of an intraocular lens (IOL) implantation, different methods of intraocular lens power calculation are used.

All major formulas currently use the measured axial length of the eye and the measured keratometric corneal power and estimate the postoperative position of the IOL. Different formulas use various methods to estimate the position of the IOL.

Most of the formulas use a constant that depends on the IOL model to estimate the position of the IOL in the eye. The name of this constant differs in different IOL power calculation formulas, such as A-constant or Surgeon factor. The estimation of the position of the IOL was improved with the refinements of this constant depending on mainly the axial length of the eye.

Some new generation formulas, such as Holladay 2 formula use also some characteristics of the anterior segment of the eye such as preoperative anterior chamber depth, lens thickness or white-to-white measurement to improve the precision of IOL calculation formulas.

Other new generation formulas use more complex mathematical and optical methods to estimate the position of the IOL in the eye, that are physically more exact, but the general problem of better estimate of the exact axial and three-dimensional position of the IOL remains.

In this connection reference is made to: Olsen T. Theoretical approach to intraocular lens calculation using Gaussian optics. J Cataract Refract Surg 1987; 13:141-145.

No method currently recognizes the role of the capsulorhexis size or shape in the estimation of the exact position of the IOL, prediction of the refractive outcome and selecting of the desired IOL.

The newer intraocular lenses such as IOL correcting spherical aberration of the eye or multifocal IOL have other optical characteristics other then the dioptric power of the IOL. These are currently commercially available only in limited modifications for particular model of IOL. Depending on the desired postoperative refractive outcome and the optical characteristics of patient's eye in the near future we might be able to select more IOL's with different optical characteristics. Even slightly incorrect placement of these IOL's that attempt to correct other optical characteristics then the dioptric power of the intraocular lens could induce other undesired optical characteristics of the eye. Thus these newer IOL's might require more precise placement of the IOL in the eye and new more precise methods of selecting the IOL are desirable.

The exact position of the IOL is difficult to estimate as it is likely influenced by the exact geometry of the intraocular lens, the exact anatomical and physiological characteristics of the patient's eye and the surgical alterations of the patient's eye. In the past when only various dioptric powers of the IOL were commercially available designed to correct the spherical error of the eye in patients with limited vision secondary to cataracts, the effect of variables other then corneal power and axial length of the eye were not recognized and of limited practical importance. Specifically the role of the capsulorhexis size or shape in the prediction of the refractive outcome and selection of the desired IOL was not recognized. With the introduction of new generation of IOL's and replacement of mild cataracts or natural lenses to correct the refractive errors of the eye, the effect of additional factors, such as the size and shape of capsulorhexis is important. The present invention improves on previous methods of selecting IOL as it recognizes the role of size and shape of capsulorhexis in achieving certain refractive outcome.

### DESCRIPTION OF THE INVENTION

The object of the invention is to describe a method of selecting an intraocular lens to be implanted into an eye to postoperatively render the eye with any chosen refractive outcome, which method should be applicable to intraocular lenses or other implanted substances placed in the proximity of remnants of an anterior capsule as for example in the capsular bag or cilliary sulcus.

Capsulorhexis is the opening of the anterior capsule of the lens made during the surgical procedure of cataract extraction. Our present invention describes that the size and shape of capsulorhexis is to be used in addition to other factors to select the type and parameters of the intraocular lens to be implanted into an eye to achieve certain refractive outcome.

The selection of intraocular lens (IOL) to be implanted into an eye to achieve desired refractive outcome is based on the knowledge of exact position and movement of an intraocular lens in the eye after the surgery. At present the selection of IOL is most commonly based on formulas calculating the power of intraocular lens from two variables of measured axial length of the eye and curvature of the corneal surface. Most formulas use also constant specific for intraocular lens derived from the geometry and optical properties of the intraocular lens. This constant can be modified by previous refractive outcomes of the surgeon. In recent years other variables describing anatomical dimensions of the eye before surgery such as preoperative anterior chamber depth, natural lens thickness, anterior segment diameter (as white-to-white measurements) were used in formulas to determine the type and parameters of the intraocular lens to be implanted into an eye. The size and shape of capsulorhexis was not used in the determination of the type and optical characteristics of intraocular lens to be selected to achieve desired refractive outcome.

The present invention improves upon prior art methods of selecting an intraocular lens as it considers in addition to other factors the size and shape of the capsulorhexis on the position of intraocular lens to postoperatively render the eye with a desired postoperative refraction or other optical characteristics.

The improved accuracy of the prediction of refractive outcome with specific IOL is attained by the method according to the invention, which considers the effect of the size and shape of the capsulorhexis on the position of the IOL in all three dimensions (as for example axial location, tilt, rotational position of an IOL) and on the movement of the intraocular lens in the eye.

Currently most intraocular lenses correct only spherical refractive errors. Intraocular lenses are recently used not only for replacement of diseased opaque lens called cataract, but also for replacement of natural lens to correct refractive errors of the eye. Some newer IOL's attempt to correct astigmatism or spherical aberration of the eye. Some newer IOL's also attempt to provide focus at distance and near in the form of some degree of accommodation, pseudoaccommodation or multifocality and thus correct presbyopia. This can be achieved by different approaches as for example multifocal IOL's or changing effective power of IOL's by its movement in the eye. Exact three dimensional position of IOL's and its movement thus become crucial for the success of correction of optical aberrations and accommodation. This requires improvements in the methods of selection of IOL to be implanted in a specific eye. The exact position of the IOL is not determined only by the characteristics of the IOL and anatomy of natural eye, but also on the anatomical changes caused by the surgery. Specifically the size and shape of the capsulorhexis does influence significantly the position and movement of the IOL in the eye and the method of the present invention requires the use of the size and shape of capsulorhexis to improve the accuracy of desired refractive outcome.

### PREFFERED EMBODIMENTS

The improved precision of desired refractive outcome based on the present invention that considers also the effect of the size and shape of capsulorhexis on the refractive outcome can be achieved by specifying the size or shape of capsulorhexis for a particular IOL to achieve desired refractive outcome by the manufacturer or expert in the field. For example we have recognized in our research that a 6 mm round centered capsulorhexis in an eye with an IOL with a 5.5 mm optic could cause 0.75 diopter difference in a postoperative refraction as compared with a round centered 5 mm capsulorhexis. A specification of 4.5 mm to 5 mm capsulorhexis with this IOL would improve the accuracy of the refractive outcome. It is recognized that particularly important is if the size of the capsulorhexis is smaller or larger compared to the size of the optic and the amount and shape of its overlap.

Alternatively the improved precision of desired refractive outcome based on the present invention can be achieved by specifying the correction factor for optical characteristics of the previously selected IOL in case that the size or shape of capsulorhexis is found to be different during the surgery to achieve desired refractive outcome. For example a selected IOL with 5.5 mm optic for particular patient is 20D IOL assuming the use of 5 mm capsulorhexis. During the surgery a 6 mm capsulorhexis is created and a correction factor of 0.5 D is used to cancel the previously selected IOL and select the IOL with 19.5 D power. It is recognized that the correction factor can be influenced by the characteristics of IOL as well as patient's eye characteristics. It is also recognized that other optical characteristics and not only power of IOL may be adjusted if the IOL with these optical characteristics are commercially available.

Alternatively the IOL can be preoperatively selected based on the formula that for a specific IOL and patient preoperative anatomical characteristics of the eye and specific size and shape of capsulorhexis calculates the predicted refractive outcome. For example for an IOL with a 5.5 mm optic and a 5 mm round centered capsulorhexis a corrective factor of +0.75 diopters is included in the formula predicting the desired refractive outcome as compared to the 6 mm capsulorhexis.

Alternatively the IOL can be selected or deselected before or during the surgery based on the achieved irregular size and shape of capsulorhexis. Certain types of IOL may be completely contraindicated with certain types of capsulorhexis as they would induce undesired optical aberrations. For example a 5.5 mm×6.5 mm eccentric oblique capsulorhexis was achieved during the surgery. The preoperative selection of multifocal IOL is cancelled. An IOL with a 6 mm optic is selected with optical characteristics that with this type of eccentric oblique capsulorhexis will achieve desired refractive outcome.

We claim:

1. A method of selecting an intraocular lens, where the size and shape of capsulorhexis of an implanted intraocular lens is used in addition to other factors to select the properties of the intraocular lens implanted in the eye to achieve certain refractive outcome.

2. The method as claimed in claim 1, wherein the size and shape are measured during the surgical procedure and based on this information the intraocular lens is selected.

3. The method as claimed in claim 1, wherein the predetermined size and shape of capsulorhexis are intentionally created surgically to achieve predetermined refractive outcome with predetermined intraocular lens.

4. The method as claimed in claim 1, wherein the property of the intraocular lens is the power of the intraocular lens.

5. The method as claimed in claim 4, wherein the power of the intraocular lens is adjusted between 0.25 to 0.75 diopters based on the size of the capsulorhexis or the overlap of the remaining anterior capsule over the optic of the intraocular lens.

6. The method as claimed in claim 1, wherein the properties of the intraocular lens are more complex optical characteristics of the intraocular lens then the dioptric power of the intraocular lens.

7. A method of selecting an intraocular lens, where the overlap of capsulorhexis over the implanted intraocular lens is used in addition to other factors to select the properties of the intraocular lens implanted in the eye to achieve certain refractive outcome.
